(19) United States
(12) Patent Application Publication
(10) Pub. No.: US 2023/0172183 A1
(43) Pub. Date: Jun. 8, 2023

(54) DEVICE AND METHOD FOR TREATING ARTWORKS AND/OR PORTABLE HERITAGE

(71) Applicant: ICM EUROPE BV, Kampenhout (BE)

(72) Inventors: Leen GYSEN, Tielt-Winge (BE); Nikolaus WILKE, Freiburg (DE); David LAINÉ, Tielt-Winge (BE); Boaz PAZ, Bad Kreuznach (DE)

(21) Appl. No.: 17/923,718

(22) PCT Filed: May 5, 2021
(86) PCT No.: PCT/IB2021/053769

§ 371 Date: Nov. 7, 2022

(30) Foreign Application Priority Data
May 5, 2020 (BE) BE2020/5299

(51) Int. Cl. A01M 1/20; A61L 2/07; A61L 2/26

(52) U.S. Cl. CPC A01M 1/2094; A61L 2/07; A61L 2/26; A61L 2202/122; A61L 2202/14

(57) ABSTRACT

Disclosed is, on the one hand, a method for killing insects and/or mold of portable heritage and/or artworks, wherein the objects to be treated are temporarily treated in a sealed housing. On the other hand, disclosed is a device for carrying out an insect and/or mould (fungus) killing treatment on portable heritage and/or artworks, including a housing for temporarily placing the heritage and/or artwork of The method and device according to this disclosure were developed in particular to protect artworks and/or portable heritage against damage (by deterioration) and thus to preserve them for a longer period.

Disclosed is a method for decontaminating and/or extracting toxic substances of portable heritage and/or artworks, where the objects to be treated are temporarily treated in a sealed housing.

### FIELD OF DISCLOSURE

The present disclosure relates, on the one hand, to a method for killing insects and/or mould of portable heritage and/or artworks, wherein the objects to be treated are temporarily treated in a sealed housing. On the other hand, the present disclosure relates to a device for carrying out an insect and/or mould (fungus) killing treatment on portable heritage and/or artworks, comprising a housing for temporarily placing the heritage and/or artwork in. The method and device according to this disclosure were developed in particular to protect artworks and/or portable heritage against damage (by deterioration) and thus to preserve them for a longer period.

The present disclosure further relates to a method for decontaminating and/or extracting toxic substances of portable heritage and/or artworks, wherein the objects to be treated are temporarily treated in a sealed housing.

### BACKGROUND

Portable (movable) heritage and/or (contemporary) artworks, such as printed cellulose materials, e.g. books and manuscripts, textile products, e.g. carpets, leather, fur, ethnographic pieces, but also furniture, musical instruments, polychromed sculptures and paintings, can be subject to deterioration. This is often the result of a variety of organisms, such as woodworm (Anobium punctatum), powderpost beetle (Lyctus brunneus), death watch beetle (Xestobium rufovillosum), silverfish (Lepisma saccharina), longhorn beetle (Hylotrupes bajulus), tobacco, carpet and biscuit beetle, carpet beetle, clothes moth, as well as funghi and (wood) fungus (wood rot).

In order to combat ageing, it is known to treat (fumigate/gasify) portable heritage and/or artworks with inert gases (nitrogen, carbon dioxide or argon) or toxic gases (phosphine, sulfuryl fluoride), in order to treat the deterioration in this way. However, such methods have the disadvantage of using chemical substances that may pose a risk to human health and the environment, and do not comply with current regulations, especially EU biocide regulations (2012). In the past, a lot of heritage and artworks were treated (preventively) with insecticides, such as naphthalene, DDT, PCP, etc., in order to eliminate or be able to eliminate the organisms harmful to the objects in question. However, this means that people who come into the vicinity of the objects treated in this way, such as museum staff and visitors, are exposed to the residues of all kinds of carcinogenic CMR substances, carcinogenic, mutagenic or toxic for reproduction, even though the use of these substances is now prohibited within the European Union. Some of the major insecticides also have corrosive properties and can cause discoloration in artworks, therefore, they are not acceptable from a conservation point of view. However, sulfuryl fluoride is a substance which has a corrosive effect and is still in use.

A non-toxic alternative for treating degraded heritage objects is anoxia or the low-oxygen method. In this method, the objects to be treated are (heritage and works of art) are stored airtight in a film with low or no oxygen permeability, reducing the oxygen concentration in the package from about 21% (normal atmosphere) to 0.1 to 0.3%. The major disadvantage of this method, however, is the long treatment time, and the difficulty in maintaining humidity, possibly resulting in condensation.

In addition, it cannot be guaranteed that the conditions will remain constant for the duration of the treatment. To better control the conditions, it is known to use a fixed anoxia chamber. An anoxia treatment takes at least 4 weeks.

US 2014/0271355 describes a method of oxidizing, sanitizing, disinfecting, and/or sterilizing a target, including ejecting a gas stream of a gaseous mixture comprising 50 to 30,000 ppm, chlorine dioxide from a gas source at a velocity of 25 to 900 ft/sec and contacting the gas stream with the target. Although it is possible with described method to completely elimination microbial viability, e.g. to kill all non-pathogenic and pathogenic spores, fungi, bacteria, and viruses, the described method is not suitable to treat portable heritage and/or artworks of high value and/or major historic value since applying the method would certainly cause damage to the fragile paint layers and/or finish layers of the artworks, as chlorine dioxide is a highly corrosive substance (kind of bleaching substance).

### SUMMARY

The present disclosure is therefore aimed at providing an effective and non-invasive method to start eliminating fungi and/or insects on portable heritage and/or artworks in an ecological way, and this in a relatively short treatment period, and without causing any damage to the treated objects during the treatment and with no effect on its sustainable preservation (portable heritage or artworks).

The object of the disclosure is achieved by a method for killing insects and/or mould of portable heritage and/or artworks, wherein the objects to be treated are temporarily treated in a sealed housing, wherein the objects to be treated undergo a heating cycle whereby they are first heated in the housing to a core temperature situated between 45° C. and 55° C. for a predetermined time at which the core temperature is maintained in the treated object which is between 60 minutes and 480 minutes, preferably between 60 minutes and 180 minutes, and that during the treatment, having a treatment time of at most 48 hours, the relative humidity in the housing has a value between 45% and 55%, and wherein together with the object to be treated, a reference object is present in the housing, the reference object being made of similar material and having similar dimensions as the object to be treated and being provided in its core with a temperature sensor. In particular, the relative humidity in the housing is being regulated in relation with the evolving temperature curve and is based on the humidity content of the treated object. Preferably, the reference object being made of similar material and having similar dimensions as the thickest diameter or cross-section of the object to be treated. More preferably, the temperature is monitored in this reference object and is representative for all objects to be treated.

Preferably, during the treatment, the relative humidity in the housing has a value located between 50% and 55%. Given that the process according to the disclosure only uses natural elements water and air, portable heritage and artworks can be preserved in an ecological manner. Moreover, the method is non-invasive, so that no damage will occur to the treated objects. With the method according to the disclosure, all organisms in all their life stages (eggs, larvae, adult insect) present in or on the portable heritage or artworks are destroyed and this significantly faster than the known methods.

In the case the method relates to killing insects and/or mold of portable heritage and/or artworks, the treatment duration, this is the duration that the portable heritage to be treated is in the closed (sealed) housing (enclosure), is maximum 48 hours, and is preferably maximum 36 hours, in particular average 24 hours.

Because during the treatment process the relative humidity in the enclosure, and consequently also in the object humidity in the objects placed in the casing, remains within the specified range, the treated heritages are safeguarded from damage, in particular damage due to the temperature increase in the housing.

The two parameters that are important for applying the method according to the disclosure are the temperature and the relative humidity in the housing and in the objects. These two parameters are adjustable. The relevant parameters are set according to the type of insect, the type of heritage or work of art (material from which it is made), and the humidity content in the object (heritage or work of art) at the start of the treatment.

In a preferred embodiment of the process according to the disclosure, the core temperature is located between 50° C. and 55° C., more particularly the core temperature is 52° C. or 53° C. By heating the heritage to be preserved until they have a core temperature of 52 to 55° C., any organisms, whatever stage in their life cycle they are in, are effectively eliminated, and this as soon as the relevant temperature in the core of the treated object has been achieved for a well-defined time. The time for which the core temperature is maintained is between 60 minutes and 480 minutes, more preferably it is at least 60 min, preferably located between 60 min and 180 min., more preferably located between 60 min and 150 min. The relevant time is preferably on average 90 minutes. The relevant time depends on the target core temperature, when the core temperature is lower, e.g. lower than 52° C. then it will be necessary for the object to maintain the lower core temperature for a longer time.

According to a more preferred embodiment of the process according to the disclosure, a cooling cycle follows the holding phase of the heating cycle. During the cooling cycle, the housing remains closed and the temperature inside the housing is gradually lowered. Also during the cooling cycle, the relative humidity in the housing remains at the preset value, usually between 45% and 55%. Preferably, the cooling phase (cycle) is 12 hours maximum.

In a particular embodiment of the process according to the disclosure, the temperature difference between the core temperature and the surface temperature is monitored during treatment. In order to measure the surface temperature, the housing is provided with temperature sensors suitable for this purpose. Monitoring of the temperature ensures an effective temperature for killing of the insects and/or fungi (mold), and avoids damages to the object due to temperature.

According to the disclosure, a reference object is present in the housing together with the object to be treated. The reference object is made of similar material and has similar dimensions as the object to be treated and is provided in its core with a temperature sensor. By using a reference object, one can determine approximately the core temperature of the object to be treated and should not damage the heritage by being able to place the temperature sensor centrally in the reference object. The reference objects are preferably round or beam-shaped. Using a reference object provides an estimate of the temperature inside the core of the actual object to be treated, so that the user can set the temperature in the housing in order to heat, maintain and cool, the core temperature of the treated object. By presetting the maximum allowed temperature difference (deltaT) between the room temperature (=surface temperature) and the core temperature inside the reference object in all three phases (heating, holding, cooling) it is ensured that the objects don't warm up too quickly and don't cool down too quickly which might put stress on them.

There is a range of prefabricated reference objects (into which probes are inserted) in different sizes which resemble the diameters of real-life museum objects. Small objects will be treated with a small diameter reference block, medium with medium, large with large etc.

The method according to the disclosure can also be used to decontaminate portable heritage and/or artworks and/or to extract (extract) toxic substances from heritage (or works of art). Indeed, in the past numerous objects in museums and collections were treated with a wide range of toxic, carcinogenic CMR substances, (carcinogenic, mutagenic, or toxic for reproduction) (naphthalene, DDT, permethrin, lindane, PCP, . . . ), either preventively or to treat active contamination by insects, microorganisms or fungi. As a result, museum staff and visitors are today exposed to the residues of these substances. In addition, the substances mentioned also have a destructive effect on the object, so that they also threaten the integrity of the heritage.

Another object of this disclosure therefor relates to a method for decontaminating and/or extracting toxic substances of portable heritage and/or artworks, wherein the objects to be treated are temporarily treated in a sealed housing, wherein the objects to be treated undergo a heating cycle whereby they are first heated in the housing to a core temperature situated between 45° C. and 55° C. for a predetermined time at which the core temperature is maintained in the treated object which is between 24 hours and 72 hours, and that during the treatment having a treatment time of at most 120 hours, preferably a treatment time of at most 96 hours, the relative humidity in the housing has a value between 45% and 55%, and in that together with the object to be treated, a reference object is present in the housing, the reference object being made of similar material and having similar dimensions as the object to be treated and being provided in its core with a temperature sensor.

In the case the method relates to decontaminating portable heritage and/or artworks, the treatment duration, this is the duration that the portable heritage to be treated is in the closed (sealed) housing (enclosure), is maximum 120 hours, and is preferably maximum 96 hours. The treatment duration depends on the kind of biocide/pesticide and concentration of it as well as the type of material the object is made of and on the maximum diameter.

Said method also comprises a cooling step and the possibility of monitoring the temperatures as disclosed in claims 4 and 5, and as described earlier. When decontaminating, the heating and cooling cycles will take a maximum of 12 hours each.

When the method is applied to extract toxic substances from a movable heritage, the method also provides for a filter step—in particular a two filter step method, one running all the time, a second one which is thermally separated and comes into effect in a second cycles phase—in which the toxic substances released during treatment are removed from the housing through a filter unit provided for that purpose. The values for treatment time, humidity and core temperature may differ from those applied in the method as described in the present set of claims which relates to the method for killing insects. Said filter step can also be provided in the method for killing insects.

Another subject matter of the present disclosure relates to a device for performing an insect and/or mold killing treatment on portable heritage and/or works of art, comprising a housing for temporarily placing the object to be treated therein, wherein the device:
air supply means for feeding air into the housing;
heating means for heating the air present in the housing between 45° C. and 60° C. preferably between 45° C. and 55° C.;
humidification means provided to give the air present in the housing a relative humidity of air located between 40% and 60%, preferably between 45% and 55%;
means for temporarily placing an object to be treated;
means for temporarily placing a reference object.

The humidity content of the air blown into the housing depends mainly on the object humidity.

Preferred embodiments of the device are described in the dependent claims. In particular, the device according to the disclosure is suitable and equipped, for applying the process according to the disclosure as described, i.e method for killing insects and/or mould and the method for biocide decontaminating.

In order to further clarify the features of the present disclosure and to indicate additional advantages and particularities thereof, a description of the process and the device according to this disclosure now follows. It will be understood that nothing in the following description can be interpreted as limiting the protection claimed for this disclosure in the claims.

### BRIEF DESCRIPTION OF THE DRAWINGS

Reference is made in this description by reference figures to the drawings attached hereto, wherein:

FIG. 1: is a top view of a device according to the disclosure;

FIG. 2: shows a treatment curve of a painting frame.

### DETAILED DESCRIPTION

The present disclosure relates to a method (process) for killing insects and/or fungi (mould) on portable (movable) heritage and/or artworks, regardless of the stage in the insect's life cycle. By application of the method, the treated objects, being portable heritage and/or artworks can remain preserved for a longer time.

The method according to the disclosure is in principle a moisture regulated thermal treatment and is extremely suitable for treating objects in paper, textiles, carpets, leather, fur, ethnographic pieces but also wood and furniture, musical instruments, polychromed sculptures and paintings, in the fight against woodworm (Anobium Punctatum), powderpost beetle (Lyctus brunneus), death watch beetle (Xestobium rufovillosum) silverfish (Lepisma saccharina), longhorn beetle (Hylotrupes bajulus), tobacco, carpet and biscuit beetle, clothes moth, but also funghi and (wood) fungus (wood rot).

The treatment method according to the disclosure includes a heating phase, a holding phase and a cooling phase. In the heating phase the whole object is heated to 45° C. to 55° C., preferably to 50° C. to 55° C., a temperature at which organisms are 100% effectively eliminated. In the holding phase, i.e as soon the desired temperature is reached in the core of the object, the temperature is held for a well-defined time and at a well-defined core temperature. In the cooling phase, the object is cooled back to ambient temperature. An average treatment cycle for killing insects and/or fungi lasts about 24 hours.

The principle behind the method is to ensure stable humidity in the treated objects by maintaining the relative humidity in a limited range of variation between 45 and 55% throughout the heating cycle until to an organism lethal temperature of 52° C. on average and cooling back to ambient temperature. A continuous monitoring via computer of the relative humidity in the housing (2) preserve objects from damage. The temperature difference between the surface of an object and the core in all three phases (heating, holding, cooling phases) is also determined in advance and monitored during treatment.

According to the disclosure, a reference object is present in the housing together with the object to be treated. The reference object is made of similar material and has similar dimensions as the object to be treated and is provided in its core with a temperature sensor. By using a reference object, one can determine approximately the core temperature of the object to be treated and should not damage the heritage by being able to place the temperature sensor centrally in the reference object. The reference objects are preferably round or beam-shaped. Using a reference object provides an estimate of the real temperature of the core of the object to be treated, so that the user can set (adapt) the temperature in the housing in order to maintain the core temperature of the treated object between 45° C. and 55° C. and can determine the predetermined time at which the core temperature is maintained.

The method according to the disclosure is an ecological technique that uses only natural elements (water and air), and compared to other methods, surpasses them in speed and effectiveness. Moreover, the treatment is non-invasive, non-toxic and 100% effective and therefore cost efficient, with continued attention and respect for the integrity of the artwork.

The method is applied in a device (1) which includes a lockable housing (2) that can be considered a humidity controlled heat chamber. In practice the treatment chamber has a capacity (content) of 30 m³ to 100 m³ and can be either permanent (fixed) or mobile (e.g. provided in a truck).

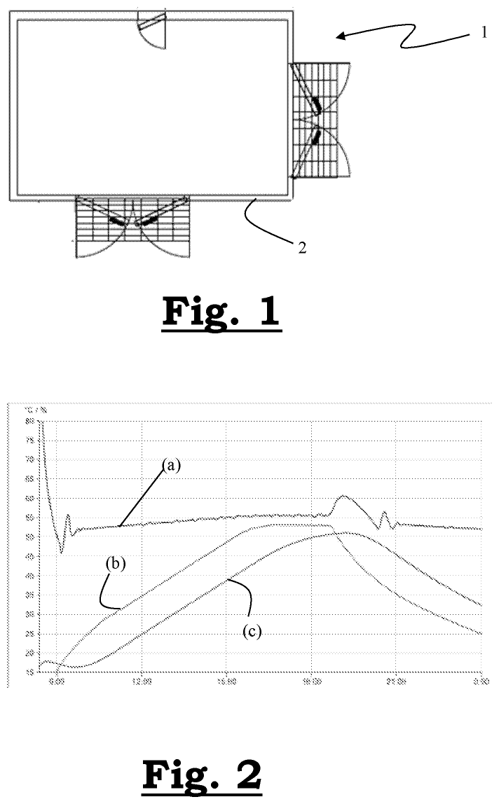

FIG. 1 shows a possible implementation form of a device (1) according to the disclosure, the device (1) comprises:
air supply means to feed air into the housing (2);
heating means for heating the air present in the housing (2) sufficiently so that an object placed in the housing (2) reaches a core temperature located between 45° C. and 55° C.;
humidifying agents provided to give the air present in the housing (2) a relative humidity located usually between 40% and 60%;
the interior of the housing (2) is also provided with means for placing the object to be treated and means for placing the reference object.

As an illustration, an enclosure (housing) may have e.g. the following dimensions: interior dimensions: 5900×4700×3600 mm. (WxDxH); outside dimensions: 6100×4900×3800 mm. (W×D×H), whereby the inner walls are provided with an antibacterial coating. The bottom is made of stainless steel. The housing is also fitted with an insulation material (e.g. 100 mm PU rigid foam) between the inner and outer walls. The housing (2) is accessible via two door wings. If necessary, an inclined plane at the level of the access doors can be provided to make it easier to bring larger objects into the interior of the housing (2).

Ultrasonic humidification is preferably used because such humidification ensures a more even distribution of humidity in the housing and the power consumption is very low.

The air is heated inside the housing to the desired temperature, the humidity is generated outside the housing (2) and added to the internal air stream. The warmed and humidified air is then blown in a continuous cycle around the room through air ducts and/or through small perforations over the entire inner side walls of the housing (2). Inside the casing are sensors that measure the relative humidity and room temperature.

In order to capture the dust particles, spores and possible residues of biocides generated during the process, the device is equipped with filter units suitable to accommodate various filter types, such as e.g. HEPA or activated carbon filters, . . . The objects to be treated, preferably heritage or artworks, after assessment by a conservator regarding temperature sensitivity of the surface layers, are placed in the housing (chamber) where they are then treated at a well-defined (core) temperature and humidity.

The two parameters (temperature and humidity) are set based on the type of insect and/or mould species, the type of object/material, and the relative humidity of the object at the start of treatment.

A treatment cycle lasts an average of 24 hours but can be slightly shorter or longer. The treatment is controlled by software via a processing unit (processor) that can also be operated remotely.

Humidification never takes place directly on the objects but outside them, e.g. in the machine room in the truck, or ultrasonically in the case of the fixed room. Sensors in reference blocks are used (placed along with the treatment chamber) to verify that the temperature is reached in the objects with the largest diameter.

The process also includes alarm thresholds that, when exceeded, cause the treatment to be stopped.

The integrity of the treated artworks and heritage is ensured by continuously monitoring and adjusting the relative humidity in the enclosure on the one hand, (when it is no longer within the specified range), and on the other hand by monitoring the difference in temperature between the surface of the treated heritage (temperature in the casing) and its core temperature, the so-called delta T. The core temperature is known by using a refence object. By defining the delta T during the ‘holding phase’ (this is the phase when the target core temperature is reached and maintained for a well-defined time), the maximum temperature in the housing and in the object can be limited, so that no overshooting can occur.

The method according to the present disclosure is currently the only one available which is completely ecological and also 100% effective for both some fungi and all insects, regardless of their life stage (adult, chrysalis, larva or egg), since the heat generated breaks down the insect's proteins. This method is also extremely fast (24 hours on average), especially compared to the known anoxia treatment which in practice takes about 4 weeks.

The method according to the disclosure is also suitable for decontaminating or at least partially extracting toxic substances from heritage and/or works of art via heating and humidity control, and this until the presence of certain substances has been reduced to an acceptable level (below a well-defined threshold). The duration of the cycles as well as the specific parameters to be used for a specific substance (toxin) depend on the toxin to be removed. When applying the method for decontaminating heritage and/or works of art, the total amount of decontamination, the total duration can be longer than the maximum of 24 hours applicable to the method of killing insects and/or fungi. Normally, the total duration should be below a maximum of 120 hours, preferably a treatment time of at most 96 hours. Furthermore, when applying this method, suitable filters should be used to capture the toxins in question.

In the example below, with reference to the curve presented on FIG. 2, the method according to the disclosure for killing insect and/or fungi is further explained.

Example: treatment of a painting frame:

In this example, a painting frame from the 17th century with polychromy and on which the presence of the organism Anobium was detected, was treated in accordance with the disclosure, and this in the presence of a reference object implemented as a thin beam-shaped wood section with a thickness corresponding to the thickness of the painting frame.

The following parameters are preset:
core temperature: 52° C.;
relative humidity (RH): 52%.

During the process the Delta T was monitored and this via the reference block, in this case these were: 10-4-12. The Delta T numbers define the maximum temperature difference between object core and object surface (=room temperature). The lower the delta T the slower the process. Depending on the thickness-section of the material to be treated a reference block is placed in the housing as well in its core is provided with a temperature sensor. Per size (diameter) a certain setting of the parameters temperature (T) and relative humidity (RH) is ideal for the process (about 24 hours), this setting can also be slightly varied through programming.

FIG. 2 shows the treatment curve where the temperature is plotted against the dwell time:
the line indicated by reference (a): indicates the stability of the relative humidity; there is a variation of ±2% with some peaks of 3%;
the line indicated by reference (b): the temperature inside the housing (room), it is up to 56° C., very stable upwards and downwards indicating a very slow and long heating up in the housing and a very slow and long cooling down in the housing;
the line indicated by reference (c): the temperature in the core of the reference block; the temperature of 52° C. in the core is maintained for 60 min. and is very stable.

Based on the information from the treatment curve, we can infer that the humidity has remained stable, a core temperature of 52° C. has been reached with 100% kill rate resulting, and this without causing any damage to the treated frame.

1. Method for killing insects and/or mould of portable heritage and/or artworks, wherein the objects to be treated are temporarily treated in a sealed housing, wherein the objects to be treated undergo a heating cycle whereby they are first heated in the housing to a core temperature situated between 45° C. and 55° C. for a predetermined time at which the core temperature is maintained in the treated object which is between 60 minutes and 480 minutes, and that during the treatment having a treatment time of at most 48 hours, the relative humidity in the housing has a value between 45% and 55%, and in that together with the object to be treated, a reference object is present in the housing, the reference object being made of similar material and having similar dimensions as the object to be treated and being provided in its core with a temperature sensor.

2. Method according to claim 1, wherein the core temperature is situated between 50° C. and 55° C.

3. Method according to claim 1, wherein the relative humidity in the housing has a value between 50% and 55%.

4. Method according to claim 1, wherein the treatment comprises also a cooling cycle which follows after the heating cycle.

5. Method according to claim 1, wherein during the treatment the temperature difference between the core temperature of the reference object and the surface temperature of the reference object is monitored.

6. Method for decontaminating and/or extracting toxic substances of portable heritage and/or artworks, wherein the objects to be treated are temporarily treated in a sealed housing, wherein the objects to be treated undergo a heating cycle whereby they are first heated in the housing to a core temperature situated between 45° C. and 55° C. for a predetermined time at which the core temperature is maintained in the treated object which is between 24 hours and 72 hours, and that during the treatment having a treatment time of at most 120 hours, the relative humidity in the housing has a value between 45% and 55%, and in that together with the object to be treated, a reference object is present in the housing, the reference object being made of similar material and having similar dimensions as the object to be treated and being provided in its core with a temperature sensor.

7. Device for carrying out an insect and/or mould treatment of portable heritage and/or artworks, comprising a housing for temporarily placing therein the object to be treated, comprising air supply means for feeding air into the housing, heating means for heating the air present in the housing, humidification means provided to give the air present in the housing a relative humidity, wherein the device further comprises a reference object which is present in the housing, the reference object being made of similar material and having similar dimensions as the object to be treated and being provided in its core with a temperature sensor.

8. Device according to claim 7, wherein the device comprises first measuring means for measuring the relative humidity in the housing, second measuring means for measuring the core temperature of the reference object, and third measuring means for measuring the surface temperature of the reference object placed in the housing.

9. Device according to claim 8, wherein the device further comprises a processing unit includes capable of processing the signals generated by the measuring means.

10. Device according to claim 7, wherein the device has a capacity located between 25 m³ and 100 m³.

11. (canceled)
